(19) United States
(12) Patent Application Publication
(10) Pub. No.: US 2025/0295508 A1
(43) Pub. Date: Sep. 25, 2025

(54) STENT GRAFT FIXATION DEVICE

(71) Applicant: Medtronic Vascular, Inc., Santa Rosa, CA (US)

(72) Inventors: Evelyn C. COLLIER, Santa Rosa, CA (US); Sri Sai Karan MUMMOJU, Santa Rosa, CA (US); Ciaran M. MCGUINNESS, Santa Rosa, CA (US); Igor HEINZE, Santa Rosa, CA (US)

(21) Appl. No.: 19/063,689

(22) Filed: Feb. 26, 2025

Related U.S. Application Data
(60) Provisional application No. 63/566,964, filed on Mar. 19, 2024.

(51) Int. Cl. A61F 2/966; A61F 2/958

(52) U.S. Cl. CPC A61F 2/966; A61F 2/958; A61F 2210/0014; A61F 2220/0016; A61F 2250/0004; A61F 2250/0069

(57) ABSTRACT

A method of deploying a fixation device to anchor a stent graft against an inner surface of a vessel includes positioning a delivery device within the stent graft that has been implanted in the vessel, wherein the fixation device is attached to the delivery device and covered by a sheath; withdrawing the sheath relative to the fixation device to expose the fixation device; and expanding the fixation device to apply an outward radial force against an inner surface of the stent graft and an inner surface of the vessel for anchoring and sealing the stent graft against the inner surface of the vessel.

### CROSS-REFERENCE TO RELATED APPLICATIONS

This application claims the benefit of U.S. Provisional Patent Application Ser. No. 63/566,964, filed Mar. 19, 2024, the entire content of which is incorporated herein by reference.

### TECHNICAL FIELD

The present disclosure relates to a prosthesis, and in particular, a fixation and sealing of prosthesis used in the repair of diseased and/or damaged sections of a hollow body organ and/or a blood vessel.

### BACKGROUND

The weakening of a vessel wall from damage or disease can lead to vessel dilatation and the formation of an aneurysm. Left untreated, an aneurysm can grow in size and may eventually rupture, potentially causing death. Aneurysms can occur anywhere throughout the circulatory system, but most commonly develop along the aorta and in blood vessels of the brain. In the aorta, aneurysms can occur in the thoracic region between the aortic arch and renal arties, as well as in the abdominal region, usually in the intrarenal area between the renal arteries and the aortic bifurcation.

Common treatment of a diseased or damaged section of a blood vessel includes open surgical replacement and endovascular aneurysm repair (EVAR). With EVAR the aneurysm is bridged with a vascular prosthesis, which is placed intraluminally. Prosthetic grafts, such as endovascular aortic grafts for aortic aneurysms, are usually designed with a fabric material attached to a metallic scaffolding or stent that self-expands or is expanded to contact the internal diameter of the vessel. The graft is held in place by the radial force of the stent and/or barbs protruding from the stent, which penetrate into the native vessel during deployment. In certain cases the stent graft may need to be deployed in complex locations, such as where the stent graft may obstruct an branch vessel and the proximal end of the graft can only be deployed a minimal distance from the branch vessel, or where the angulation, curvature, or diameter of the vessel affects the risk of collapse, migration, or failure of aneurysm exclusion due to improper fixation or sealing of the graft against the aortic wall. Further complexity may result from the shape and size of the aneurysm. This complexity can result in considerable time to position, fixate, and ensure a proper seal of the stent graft. Additionally, stent grafts may exhibit migration or endoleak and require augmented radial fixation and/or sealing to regain or maintain aneurysm exclusion. This again can require considerable time to repair or replace the stent graft.

Therefore, there is a desire in the art to reduce the time required for the fixation and sealing of stent grafts at a desired location and to provide a means for preventing and/or repairing migration or endoleaks of a deployed stent graft.

### SUMMARY

Embodiments according to the present invention address aneurysm repair and in situ positional stability of a device used for aneurysm repair. Specifically, embodiments according to the present invention provide systems and methods for fixating and scaling a prostheses in a diseased and/or damaged section of a hollow body organ, blood vessel, and/or bodily lumen using a fixation device. Additionally, the fixation device provide a means for repairing and maintaining prostheses that has experienced migration or endoleak. Although the specification provides specific configurations for use in abdominal and thoracic locations, stent grafts according to the invention are readily applicable to uses in other aneurysmal locations or associated branch vessels. Fixation devices according to the present invention can also be used in modular components of a stent graft, including junctions between a main body of a stent graft and other stent graft extensions such as cuffed braches or fenestrations.

In embodiments, a method of deploying a fixation device to anchor a stent graft against an inner surface of a vessel includes positioning a delivery device within the stent graft that has been implanted in the vessel, wherein the fixation device is attached to the delivery device and covered by a sheath; withdrawing the sheath relative to the fixation device to expose the fixation device; and expanding the fixation device to apply an outward radial force against an inner surface of the stent graft and an inner surface of the vessel for anchoring and scaling the stent graft against the inner surface of the vessel.

In embodiments, the expanding step includes a self-expansion of the fixation device, wherein the fixation device is composed of a shape memory material.

In embodiments, the expanding step includes a balloon expansion, wherein the fixation device is expanded by inflation of a balloon connected to the delivery device.

In embodiments, the expanding step includes mechanically adjusting the fixation device, wherein the delivery device includes a plurality of adjusting members connected to the fixation device and actuation of the plurality of adjusting members expands the fixation device radially by translating the fixation device along a longitudinal axis to shorten the fixation device longitudinally.

In embodiments, the expanding step includes mechanically adjusting the fixation device by actuation of the plurality of adjusting members shrinks the fixation device radially by translating the fixation device along a longitudinal axis to elongate the fixation device longitudinally for repositioning the fixation device.

In embodiments, the expanding step includes removing an external sheath surrounding the fixations device before the fixation device applies the outward radial force against the inner surface of the stent graft.

In embodiments, the fixation device includes a cylindrical metal mesh and the plurality of adjusting members are connected to an inner surface of the fixation device.

In embodiments, the withdrawing step includes recapturing the fixation device by advancing the sheath relative to the fixation device to cover the fixation device for repositioning the fixation device.

In embodiments, the fixation device includes an anchor for penetrating the inner wall of the vessel.

In embodiments, the position step includes inflating a balloon connected to the delivery device for aligning the fixation device within the stent graft such that the anchor is perpendicular to the inner surface of the vessel when the fixation device is expanded.

In embodiments, a system for fixating and scaling a prosthesis against an inner surface of a vessel, the system includes a delivery device having a distal end, wherein the delivery device is configured for insertion into a patient's vasculature and advancement of the distal end through the patient's vasculature and into a stent graft that has been deployed in the vessel; a fixation device attached to the distal end of the delivery device and configured for expanding radially and applying outward radial force against an inner surface of the stent graft for sealingly anchoring the stent graft to the vessel; and a plurality of anchors disposed on an outer surface of the fixation device that are configured to penetrate the inner surface of the vessel.

In embodiments, the fixation device includes a frame having a shape memory configuration with a diameter larger than the diameter of a location within the vessel, wherein the fixation device is configured to be compressed and then expanded such that the plurality of anchors are perpendicular to the inner surface of the vessel.

In embodiments, the frame of the fixation device is composed of nitinol.

In embodiments, the fixation device includes an inner cover disposed on an inner surface of the fixation device and an outer cover disposed on the outer surface of the fixation device, wherein the inner and outer covers are configured to seal the stent graft against the inner surface of the vessel and guide flow through the stent graft.

In embodiments, the fixation device includes a proximal end and a distal end distal to the proximal end, and wherein the plurality of hooks are disposed on at the proximal end and the distal end of the fixation device.

In embodiments, the fixation device includes a proximal end and a distal end distal to the proximal end, and wherein the fixation device includes a unitary body having a low profile such that a proximal end and a distal end of the fixation device are in close proximity to each other.

In embodiments, a system for fixating and sealing a prosthesis against an inner surface of a vessel includes a balloon configured to inflate and deflate, wherein the fixation device is disposed on an outer surface of the balloon such that the fixation device expands radially when the balloon is inflated, and wherein the fixation device is configured to remained expanded after the balloon is deflated.

In embodiments, the delivery device further includes an adjustment member connected to the fixation device, wherein the fixation device includes a cylindrical metallic mesh frame, and wherein actuation of the adjustment members expands the fixation device radially by translating the fixation device along a longitudinal axis to shorten the fixation device longitudinally.

In embodiments, a system for fixating and sealing a prosthesis against an inner surface of a vessel includes an external sheath surrounding the fixation device and configured to be removed from contact with an outer surface of fixation device after expansion of the fixation device, and wherein the external sheath is configured to prevent the plurality of hooks from engaging the inner surface of the vessel.

In embodiments, the plurality of hooks are disposed between a proximal end and a distal end of the fixation device.

### BRIEF DESCRIPTION OF THE DRAWINGS

FIG. 1 is an artist's rendering of a cross section of an aorta showing an aneurysm being bridged by a stent graft;

FIG. 2 is a cross sectional side view illustrating a delivery system delivering a fixation device inside of the stent graft of FIG. 1;

FIG. 3 is a cross sectional side view illustrating an embodiment of the fixation device shown in FIG. 2;

FIG. 4 is a perspective view illustrating the fixation device of FIG. 3 according to another embodiment;

FIG. 5 is a perspective view illustrating the fixation device of FIG. 3 according yet another embodiment;

FIG. 6 is a cross sectional side view illustrating an embodiment of the fixation device shown in FIG. 2;

FIG. 7 is a top perspective view illustrating the embodiment of the fixation device shown in FIG. 6;

FIG. 8 is a cross sectional side view illustrating an embodiment of the fixation device shown in FIG. 2, wherein the fixation device is expanded by a balloon;

FIG. 9 is a cross sectional side view illustrating an embodiment of the fixation device shown in FIG. 2, wherein the fixation device is expanded by mechanical adjustment;

FIG. 10 is a cross sectional side view illustrating the fixation shown in FIG. 9 having a first length;

FIG. 11 is a cross sectional side view illustrating the fixation shown in FIG. 9 having a second length; and

FIG. 12 is a cross sectional side view illustrating the fixation shown in FIG. 9 having a third length.

### DETAILED DESCRIPTION

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting, but merely as a representative bases for teaching one skilled in the art to variously employ the embodiments. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical application. Various combinations and modifications of the features consistent with the teachings of this disclosure, however, could be desired for particular applications or implementations.

“A”, “an”, and “the” as used herein refers to both singular and plural referents unless the context clearly dictates otherwise. By way of example, “a processor” programmed to perform various functions refers to one processor programmed to perform each and every function, or more than one processor collectively programmed to perform each of the various functions.

Directional terms used herein are made with reference to the views and orientations shown in the exemplary figures. A central axis is shown in the figures and described below. Terms such as “outer” and “inner” are relative to the central axis. For example, an “outer” surface means that the surfaces faces away from the central axis, or is outboard of another “inner” surface. Terms such as “radial,” “diameter,” “circumference,” etc. also are relative to the central axis. The terms “front,” “rear,” “upper” and “lower” designate directions in the drawings to which reference is made.

Unless otherwise indicated, for the delivery system the terms “distal” and “proximal” are used in the following description with respect to a position or direction relative to a treating clinician. “Distal” and “distally” are positions distant from or in a direction away from the clinician, and “proximal” and “proximally” are positions near or in a direction toward the clinician. For the stent-graft prosthesis, “proximal” is the portion nearer the heart by way of blood flow path while “distal” is the portion of the stent-graft further from the heart by way of blood flow path.

The following detailed description is merely exemplary in nature and is not intended to limit the invention or the application and uses of the invention. Although the description is in the context of treatment of blood vessels such as the aorta, coronary, carotid and renal arteries, the invention may also be used in any other body passageways where it is deemed useful.

Methods and systems for stabilizing and treating an aneurysm include deploying a sectional exclusion device, such as a stent graft, in the flow lumen of a blood vessel to span and seal off the aneurysmal location from further blood flow while acting as a conduit to direct blood flow past the aneurysmal site. To prevent occlusion of arteries (e.g., the common iliac artery, external iliac artery, internal iliac artery, renal arteries, or the like) or other branch vessels a bifurcated or fenestrated stent graft may be used whereby other stent graft extensions are sealingly attached to a main body of the stent graft. The systems and methods are described herein with respect to treating aortic aneurysms, however, one of ordinary skill in the art will understand that the systems and methods may be used in other areas of the vasculature and/or for other pathologics.

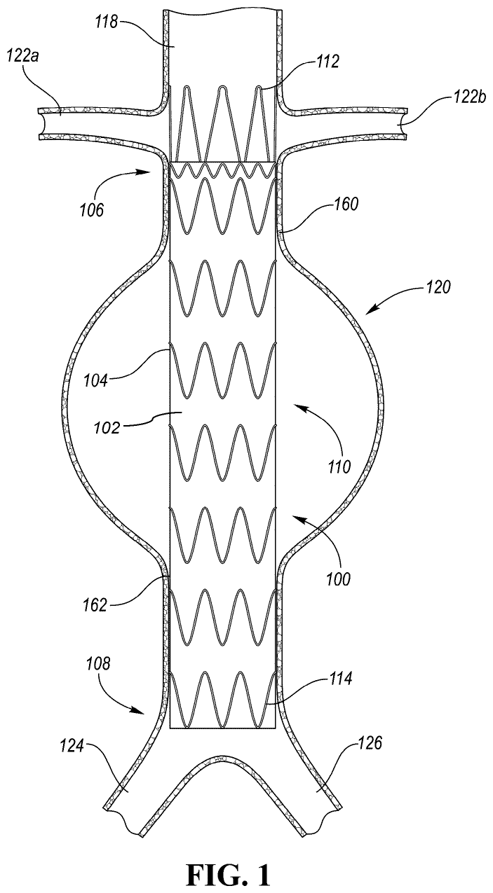

Referring to FIG. 1, there is shown an exemplary depiction of a stent graft 100 deployed in a blood vessel 118. The stent graft 100 generally includes a tubular graft 102 and one or more stents 104 for supporting and expanding the graft 102. The graft 102 may be formed from any suitable graft material, for example but not limited to, a low-porosity woven or knit polyester, DACRON material, expanded polytetrafluoroethylene, polyurethane, silicone, or other suitable materials. The graft material may also be a natural material such as pericardium or another membranous tissue such as intestinal submucosa. The one or more stents 104 are generally radially-compressible and expandable, and are coupled (e.g., via stitching) to the material of the graft 102 for supporting the graft 102. Stent grafts 100 generally include a proximal end 106, and a distal end 108, and a body 110 therebetween. The proximal end 106 may have a proximal stent 112, and the distal end 108 may have a distal stent 114. The proximal stent 112 may extend outside of the graft 102 (e.g., a bare stent), as shown, and may also be generally described as an anchor stent or crown stent, configured to anchor to inner walls of the vessel 118. In other embodiments, the distal stent 114 may also be a bare stent, however, the proximal and/or distal stents may also not extend past the graft material (e.g., a covered seal stent). The vessel 118 show in FIG. 1 is the aorta and the stent graft 100 bridges an aneurysm 120 to guide blood flow through the stent graft 100 to healthy regions of the vessel 118 downstream of the aneurysm 120. The aneurysm 120 is shown as an abdominal aneurysm, however, the present disclosure also applies to thoracic, arch, or peripheral aneurysms. The blood flow is guided through the stent graft 100 by providing a seal between the inner wall of the vessel 118 and the graft 102 at the proximal end 106 and distal end 108 of the stent graft 100.

The stent graft 100 shown in FIG. 1 is deployed in the abdominal aorta region where there is no occlusion of important arteries or other branch vessels such that a bifurcated or fenestrated stent graft is not required. For example, the stent graft 100 is deployed such that proximal stent 112 is adjacent to the renal arteries 122a, 122b and the distal stent 114 is adjacent to the right common iliac artery 124 and the left common iliac artery 126. In the embodiment shown, there is sufficient healthy landing zone at the distal end 162 of the aneurysm that a bifurcated stent graft is not required. However, as will be appreciated by the discussed herein, the stent graft 100 could be a bifurcated or fenestrated stent graft deployed in other regions of the aorta.

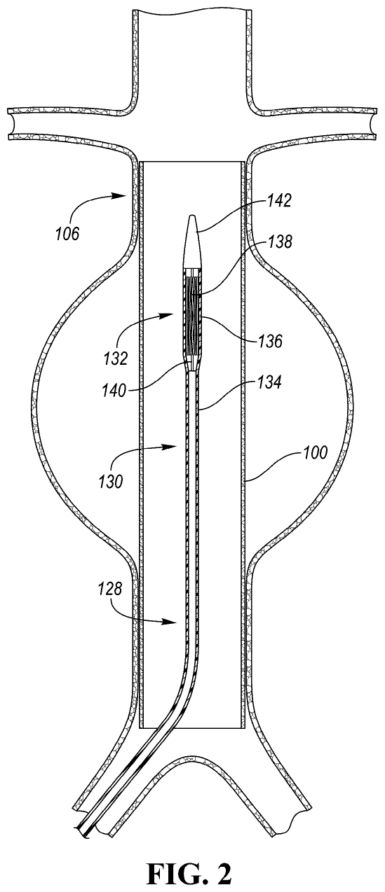

FIG. 2 illustrates an embodiment of a delivery system 128 positioned inside of the stent graft 100. The delivery system 128 generally comprises a catheter portion 130, a distal portion 132, and a control handle portion (not shown). The catheter portion 130 may be a suitable length and size so as to permit a controlled delivery of the distal portion 132 to the desired location of the aorta (e.g., the catheter portion 130 is configured for insertion of the delivery system 128 into a femoral or radial artery and delivery of the distal portion 132 to the abdominal aorta including the suprarenal and infrarenal aorta, the thoracic aorta including the descending aorta, aortic arch, and ascending aorta, or the like). The distal portion 132 may be configured for mounting an implantable device (e.g., the fixation device 138) for delivery to the desired location and may further be configured to allow the expansion of the implantable device for effective deployment thereof, as further discussed below. The control handle portion may control the movement of the catheter portion 130 to position the distal portion 132 in the desired deployment location and may also control the deployment and expansion of the implantable device for case of delivery and withdrawal of the delivery system 128 through a patient's vasculature. It can be appreciated that during delivery and/or deployment of the implantable device, portions of the delivery system 128 (e.g., the catheter portion 130 and/or the distal portion 132) will be required to be advanced through the patient's vasculature, including tortuous and/or narrow vessels 118 or lumens. Therefore, it is contemplated that the delivery system 128 is designed with portions having a suitable profile with sufficient flexibility and stiffness.

The catheter portion 130 comprises an outer shaft 134 that may extend from the control handle portion and be operatively connected with the control handle portion so as to be moveable by operation of the handle control portion. The outer shaft 134 may surround one or more shafts and may include one or more lubricous inner layers (such as high density polyethylene HDPE or Polytetrafluoroethylene PTFE), one or more braided stainless steel middle layers, and one or more flexible plastic outer layers, such as Pebax 7233, Pebax 6333,Nylon 12, Vestamid ML24, or the like. The outer shaft 134 may be connected to a sheath 136 (e.g., a sheath, or the like).

In some embodiments, the sheath 136 may be integrally formed as an extension of the outer shaft 134. In other embodiments, the sheath 136 may be a separate component from the outer shaft 134. The sheath 136 may be configured to retain a fixation device 138 in a radially collapsed configuration for delivery to the desired deployment location, as will be described in more detail below. Generally, axial movement of the outer shaft 134 results in a longitudinal translational movement of the sheath 136 proximally from its distalmost position away from the distal portion 132, thereby exposing the fixation device 138. In certain embodiments, the control handle portion may control the advancement and withdrawal of the sheath 136. The sheath 136 may be tapered from a smaller diameter adjacent to the outer shaft 134 to a larger diameter distal to the outer shaft 134, or vice versa, or may have a constant diameter similar to or the same as the outer shaft 134. The sheath 136 may be formed from any material or material composition suitable to retain the fixation device 138 (e.g., a polymer material or a combination of polymer materials, a metal or metal alloy for rigidity, or the like, or a combination or sub-combination thereof). The sheath 136 may also include an inner or outer lining that can be formed of a polymer or a combination of polymer materials such as high density polyethylene HDPE, Polytetrafluoroethylene PTFE, or the like.

The delivery system 128 further includes an inner shaft 140 disposed within the outer shaft 134 and the sheath 136. In some embodiments, the inner shaft 140 defines a lumen such that the delivery system 128 may be slidingly disposed and tracked over a guidewire (not shown) for advancement of the delivery system 128 through the patient's vasculature to the desired deployment location. The inner shaft 140 may be coupled to a distal tip 142 disposed adjacent to the sheath 136 and distal to the catheter portion 130. The distal tip 142 may be a frustoconical shape or tapered shape that decreases linearly in diameter and can be formed of a soft polymeric material allowing for engagement with tissue of a vascular system during insertion, withdrawal, and maneuvering (e.g., a flexible nosecone, or the like). The distal tip 142 may have a lumen that aligns with the lumen of the inner shaft 140 such that these components are in communication with each other in order to establish a continuous lumen for a guidewire. In some embodiments, the inner shaft 140 may be axially translatable independent of the outer shaft 134, such that the distal tip 142 is moveable independent of the outer shaft 134.

In an embodiment, the inner shaft 140 is configured to receive the fixation device 138 on a distal end of the inner shaft 140 and whereby the sheath 136 compressively retains the fixation device 138 on the distal end of the inner shaft 140. In other words, the sheath 136 surrounds and constrains the fixation device 138 in a radially compressed or delivery configuration. During deployment of the fixation device 138, the sheath 136 is proximally retracted with respect to the fixation device 138 to expose the fixation device 138. The sheath 136 may be retracted incrementally until the fixation device 138 is fully exposed. The sheath 136 may also be advanced to cover the fixation device 138 prior to the fixation device 138 being fully exposed in order to recapture the fixation device 138 (e.g., the sheath 136 may be advance to re-cover and retain the fixation device 138 in the event that the delivery system 128 must be repositioned or reoriented). In some embodiments, the inner shaft 140 may be operatively connected to the control handle portion so as to be relatively fixed with respect to the outer shaft 134 and the sheath 136 so the relative movement of the sheath 136 with respect to the inner shaft 140 provides for a controlled covering or exposing of the fixation device 138. In some embodiments, the inner shaft 140 may be axially translatable independent of the outer shaft 134, such that the distal tip 142 is moveable independent of the outer shaft 134. In other embodiments, the delivery system 128 and/or the fixation device 138 include one or more markers to aid in the position of the fixation device 138 (e.g., a radiopaque material, or the like, so that the location and/or orientation of the fixation device 138 can be perceived by an x-ray or other radiation transmission during deployment).

It is contemplated that the fixation device 138 may be attached or secured to the delivery system 128 by other means. For example, an attachment member (not shown) may retain a segment of the fixation device 138, wherein the attachment member is spaced apart from the distal tip 142, adjacent to the catheter portion 130 and the distal portion 132, or the like. When the sheath 136 is proximally retracted beyond the attachment member, the fixation device 138 may no longer be retained by the attachment member. In other embodiments, the delivery system 128 may have a proximal and/or distal capture mechanism for at least partially retaining the fixation device 138 in a compressed or retained configuration during deployment. For example, proximal or distal stent crowns of the fixation device 138 may be held on pins, nubs, or other protrusions on the inner shaft 140 or a component attached thereto and a sleeve may be configured to hold the crowns between the sleeve and the inner shaft 140 until the sleeve is removed. In one example, the sleeve may be attached to the distal tip 142 and distal advancement of the inner shaft 140 may cause the sleeve to release the crowns after the sheath 136 has been proximally retracted, thereby allowing the proximal end 150 of the fixation device 138 to be held by the delivery system after a more distal portion 152 thereof has been released. A similar system may also/alternatively be employed on the distal end 152 to hold the distal end 152 even after the sheath 136 has been retracted past the distal end 152 of the fixation device 138. In other embodiments, instead of crowns of the fixation device 138 being held, there may be one or more extensions, paddles, tethers, etc. extending from the proximal or distal end 150, 152 of the fixation device 138 which may be held or attached to components of the delivery system 128 (e.g., inner shaft 140) and released when desired. For example, a paddle attached to the fixation device 138 may be received in a pocket or opening with a complimentary shape in the inner shaft 140 or a component attached thereto (e.g., a spindle). When, for example, a sleeve attached to the distal tip 142 is advanced, the paddle may be released from the pocket and the fixation device 138 is released from the delivery system 128 (at least at that end).

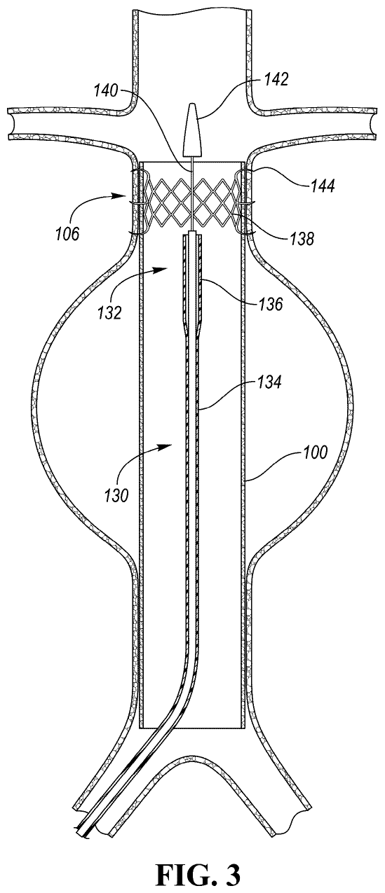

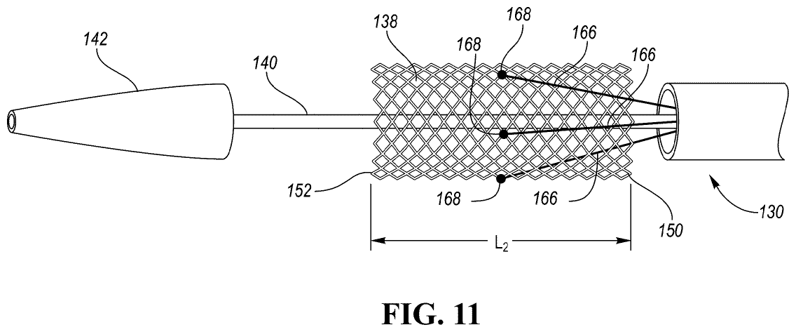

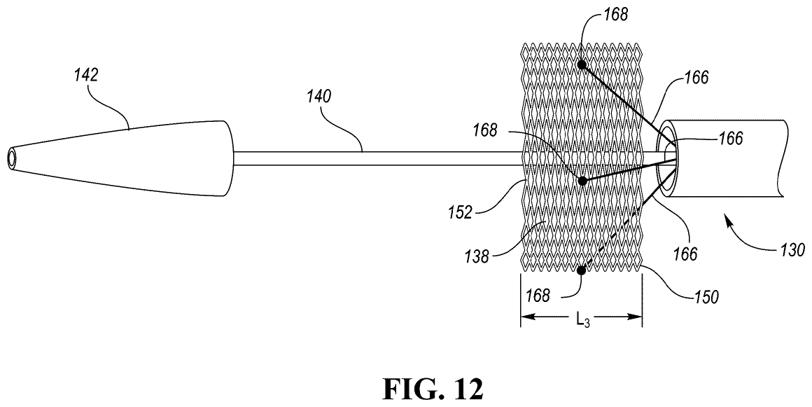

After the sheath 136 is proximally retracted, and any optional capture mechanism is released, the fixation device 138 can be expanded from the delivery configuration to an expanded deployed configuration. Referring to FIGS. 3-12, the fixation device 138 may generally be an annular structure (e.g., a circular, cylindrical, or ring-shaped, or the like) with a central lumen 146 extending therethrough. An annular structure is preferable so that the fixation device 138 can substantially mimic the shape of the stent graft 100 and the vessel 118 in order for an outer surface 148 of the fixation device 138 to be in close contact or in close proximity to the stent graft 100 and the vessel 118 when the fixation device is in the expanded deployed configuration. While in the expanded deployed configuration the fixation device 138 fixates and seals the stent graft 100 to the inner wall of the vessel 118 via an outward radial force and/or penetration into the inner wall of the vessel 118.

Referring to FIGS. 3-9, in some embodiments, the fixation device 138 includes a plurality of anchors 144 (e.g., barbs, prongs, or the like) for further securing and sealing the stent graft 100 to the inner wall of the vessel 118. The plurality of anchors 144 are configured for piercing through the graft 102 and into the inner wall of the vessel 118. As previously discussed, a fixation device with an annular structure is preferable so that the plurality of anchors 144 can be aligned perpendicular to the inner wall of the vessel 118 when piercing the inner wall of the vessel 118. The perpendicular insertion of the plurality of anchors 144 provides for a more secure anchoring of the plurality of anchors 144 into the vessel 118. The plurality of anchors 144 may be disposed at a proximal end 150 or a distal end 152 of the fixation device 138, at a point between the proximal and distal ends 150, 152 (e.g., a midpoint), or the like, or a combination or sub-combination thereof. The anchors 144 may be disposed around the entire circumference of the fixation device 138, for example, evenly spaced about the circumference.

FIGS. 3 illustrates an embodiment of the fixation device 138 being deployed adjacent to the proximal end 106 of the stent graft 100. In other embodiments, the fixation device 138 may also or alternatively be deployed at the distal end 108 of the stent graft 100. In some embodiments, the fixation device 138 may be deployed only in a healthy landing zone of the aorta (e.g., a short neck), not in an aneurysmal region. The fixation device 138 may be self-expanding and composed of a shape memory material (e.g., nickel-titanium alloy (nitinol), stainless steel, a pseudo-clastic metal, various polymers, or a so-called super alloy, which may have a base metal of nickel, cobalt, chromium, or other metal, or other suitable material) that returns the fixation device 138 from the radially compressed or delivery configuration to the expanded deployed configuration. For example, after the sheath 136 is proximally retracted and the fixation device 138 is exposed, the fixation device 138 will begin to expand radially to be in apposition to the inner wall of the vessel 118. The outward radial force of the fixation device 138 fixates the stent graft 100 to the inner wall of the vessel 118 (e.g., piercing the vessel 118 wall) to secure the positioning of the stent graft 100 (e.g., preventing migration of the stent graft 100, or the like). The outward radial force of the fixation device 138 also assists in scaling the stent graft 100 against the inner wall of the vessel to prevent endoleak. Accordingly, the deployment of the fixation device 138 can be used to maintain or regain aneurysm exclusion.

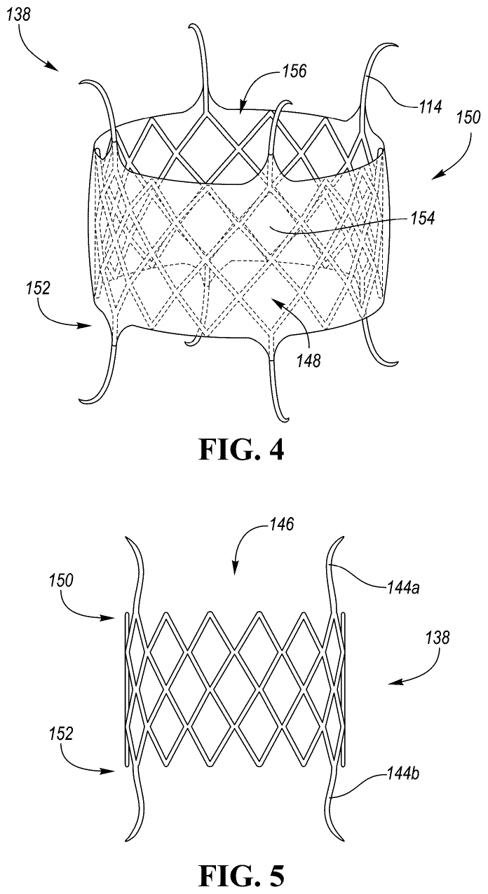

Referring to FIG. 4, an embodiment of the fixation device 138 includes an optional cover or coating 154 over an inner surface 156 or the outer surface 148 of the fixation device 138. The cover 154 may be disposed over an entirety or a portion of the inner or outer surfaces 156, 148. The cover 154 may be composed of a fabric, polymer, or previously described material for the graft 102, or the like for preventing damage to the vessel 118 or providing for an increased grip/resistance between the outer surface 148 of the fixation device 138 and either the stent graft 100 or vessel 118. The cover 154 may also be configured to guide the flow of blood, such as by sealing the stent graft 100 against the inner wall of the vessel to prevent endoleak or directing the flow through the central lumen 146. A fixation device 138 with a cover 154 may be used to extend a sealing zone of a stent graft 100 either proximally or distally by only partially overlapping the graft 102 material of the stent graft 100 (as opposed to being fully within the stent graft 100, as shown in FIG. 3).

As previously mentioned, the fixation device 138 may be recaptured by the sheath 136 prior to being fully exposed. For example, the sheath 136 may be advanced to re-cover and retain the fixation device 138 in the event that the delivery system 128 must be repositioned or reoriented. In this case, sheath 136 is configured to overcome the self-expanding nature of the fixation device 138 and radially compress the exposed portion of the fixation device 138.

Referring to FIG. 5, an embodiment of the fixation device 138 includes a plurality of anchors 144a, 144b disposed at the proximal end 150 and the distal end 152 of the fixation device 138, respectively. The embodiment shown does not have a cover 154 (e.g., a bare frame), but could in an alternate embodiment. The plurality of anchors 144a are configured to not penetrate the tissue of the inner wall of the vessel 118. Because the plurality of anchors 144a do not penetrate the tissue of the inner wall of the vessel 118 the fixation device 138 can be recapture by the sheath 136 even if the plurality of anchors 144a have been exposed. The plurality of anchors 144b disposed at the distal end 152 of the fixation device 138 can be configured to penetrate or not penetrate the tissue of the inner wall of the vessel 118. In some embodiments, the plurality of anchors 144 may be configured so as to not penetrate the inner wall of the vessel 118 until the fixation device 138 is fully exposed. Anchors 144 not configured to penetrate the graft 102 or vessel 118 may have a blunted, rounded, or atraumatic tip or may curve radially inward at the distal tip to avoid penetrating. They may also/alternatively have a coating or covering of a softer material, such as fabric or polymer.

As illustrated in FIGS. 3-5, the fixation device 138 may have a stent or wire frame. In the embodiments shown, the frame has a diamond cell structure with three rows of cells. The shape and/or number of rows may vary, for example, there may be fewer or a greater number of rows (e.g., any number or internal range between 1-10). The proximal and distalmost tips of the cells may be referred to as crowns. In some embodiments, such as shown, some anchors 144 may extend from the crowns. Points where three or more cells meet may be referred to as nodes. Anchors 144 that are disposed between the proximal and distal ends 150, 152 of the fixation device 138 may extend from a node. Anchors 144 at the proximal or distal ends 150, 152 may also extend from nodes that are disposed between crowns. Anchors 144 may also extend from locations other than crowns or nodes, such as from a strut or leg of the cells. In the embodiment shown in FIG. 3, there are three rows of anchors 144 and in the embodiment of FIGS. 4-5 there are two rows of anchors 144. However, this is not intended to be limiting and there may be one, two, three, four, five, or more rows of anchors 144, or any sub-range therein. The anchors 144 shown in FIGS. 4-5 are shown extending axially beyond the frame of the fixation device 138, however, in other embodiments the anchors 144 may extend within the height of the frame.

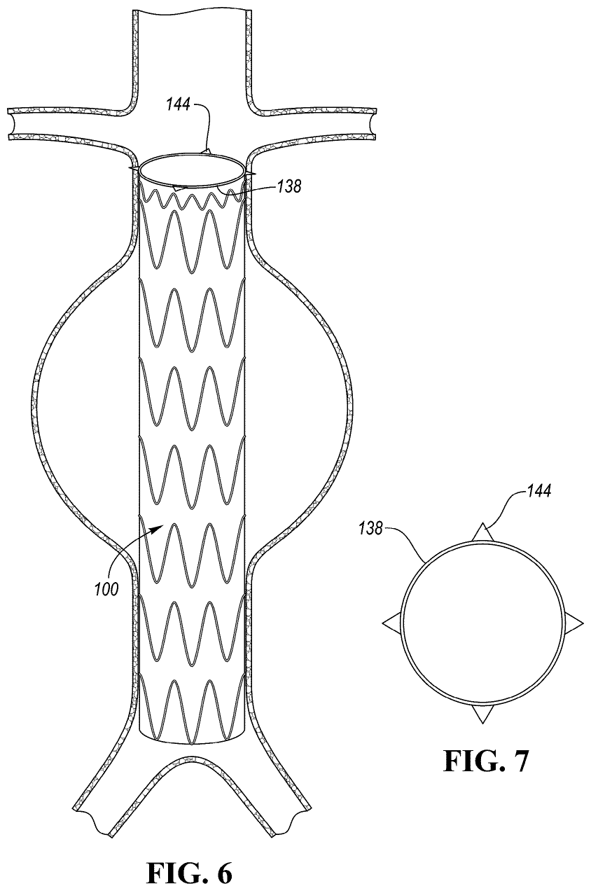

As illustrated in FIGS. 6 and 7, an embodiment of the fixation device 138 may be shaped as a substantially unitary body that resembles a ring or halo type structure. The slim, low profile, or narrow unitary body allows the fixation device 138 to be precisely deployed in various locations, such as adjacent to the proximal or distal ends 106, 108 of the stent graft 100, a proximal end 160 or distal end 162 of the aneurysm 120, or adjacent branch vessels like the renal arteries 122a, 122b, or adjacent a branch coupling or fenestration in the stent graft 100, or the like. In addition, a plurality of fixation devices 138 may be deployed in close proximity without interfering with each other. In embodiments with a radiopaque marker, the fixation devices 138 can also be used to identify various segments of the stent graft 100, the vessel 118, or the aneurysm 120.

It is also contemplated that the fixation device 138 may be used to secure, seal, or attach a branch stent graft to a fenestration or aperture of the stent graft 100. For example, where a branch stent graft has been inserted into the fenestration of stent graft 100, the fixation device 138 can be deployed to couple or attach the branch stent graft to the stent graft 100 where the two overlap by supplying an outward radial force, which presses the branch stent graft and the stent graft 100 together against the inner wall of the vessel 118, by piercing both the graft material of the branch stent graft and the graft 102 of the stent graft 100, or the like, or a combination or sub-combination thereof. Alternatively, when securing two stent grafts 100 to each other, non-piercing embodiments may be used to reduce the possibility of endoleaks. Use of the fixation device 138 in this way permits the repair or maintenance of the coupling location of the branch stent graft and the stent graft 100 without requiring replacement of either of the prostheses.

As described above, the embodiment of fixation device 138 in FIGS. 6-7 may be a ring-like structure, which may be substantially planar or two-dimensional. Stated another way, the height of the fixation device 138 may be substantially the same as the thickness of the wire forming the annular structure. In the embodiment shown, the anchors 144 may be triangular shaped, however, other shapes are contemplated such as hooks, barbs, tines, etc. In addition, while four equally spaced anchors 144 are shown (e.g., 90 degrees apart), greater or fewer anchors 144 may be present, such as 2-10 anchors or any sub-range therein. As described above, fixation device 138 may include capture features to assist with a controlled and accurate deployment, such as extensions, paddles, tethers, etc. extending proximally and/or distally from the fixation device 138 which may be held or attached to components of the delivery system 128 and released during deployment. These features may be the only portions of the fixation device 138 extending out of the plane of the ring.

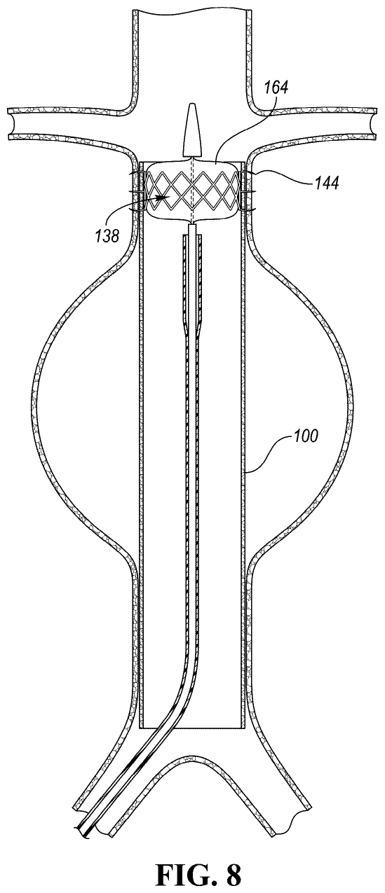

As illustrated in FIG. 8, an embodiment of the fixation device 138 (e.g., any fixation device 138 disclosed herein) may be expanded from the compressed delivery configuration to the expanded deployed configuration by a balloon 164. The balloon 164 may be positioned within the fixation device 138 (e.g., within the central lumen 146) and may surround the inner shaft 140 such that the fixation device 138 is operatively connected to the balloon 164 when the fixation device 138 is in the compressed delivery configuration. During deployment, the fixation device 138 is positioned in the desired location and the balloon 164 is inflated to fill the volume of the stent graft 100 and/or vessel 118 in order to radially expand the fixation device 138. Once the fixation device 138 has been fully expanded by the balloon 164, the balloon 164 may be deflated and removed from the central lumen 146 of the fixation device 138. The fixation device 138 is configured to maintain its expanded deployed configuration and resist constriction of the vessel 118 after expansion. The fixation device 138 may be composed of a metal mesh, a polymer including silicone, or a combination of suitable materials. The fixation device 138 may be a cylindrical structure having a mesh network, lattice, braids, or the like.

In some embodiments, the balloon 164 may be connected to the catheter portion 130, adjacent to the distal portion 132, or be connected adjacent to the distal tip 142 to assist in the alignment of the fixation device 138 during deployment. In yet further embodiments, a plurality of balloons 164 may be used to assist in the alignment of the fixation device 138.

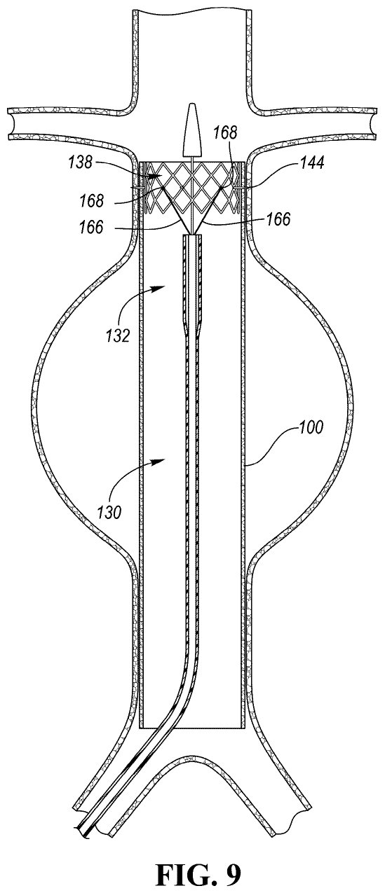

As illustrated in FIG. 9, an embodiment the fixation device 138 (e.g., any fixation device 138 disclosed herein) may be expanded from the compressed delivery configuration to the expanded deployed configuration by mechanical adjustments. The delivery system 128 includes an adjustment member 166 (e.g., surgical threads, wires, rods, tubes or other mechanical elements) that is connected to the fixation device 138 at an attachment point 168 and operatively connected to the control handle portion. The adjustment member 166 may extend from the control handle to the distal portion 132 (e.g., from within the outer shaft 134). The delivery system 128 may be configured to prevent the adjustment member 166 from becoming tangled or twisted within the catheter portion 130 and/or the distal portion 132 (e.g., the adjustment member 166 may be housed within a separate lumen within the catheter portion 130, coupled to a component that maintains the adjustment member 166 in a substantially linear configuration, or the like).

The adjustment member 166 and/or attachment point 168 may be connected to the inner or outer surface 156, 148 of the fixation device 138 and may also be connected adjacent to the proximal end 150, the distal end 152, or at a point between the proximal and distal ends 150, 152 of the fixation device 138, or the like, or a combination or sub-combination thereof. In some embodiments, the delivery system 128 includes a plurality of adjustment members 166 that are connected to the fixation device 138 at a plurality of attachment points 168 respectively. In other embodiments, the adjustment member 166 is connected to the fixation device 138 at the plurality of attachment points 168 (e.g., the adjustment member is interwoven among the mesh network of the fixation device 138, or the like).

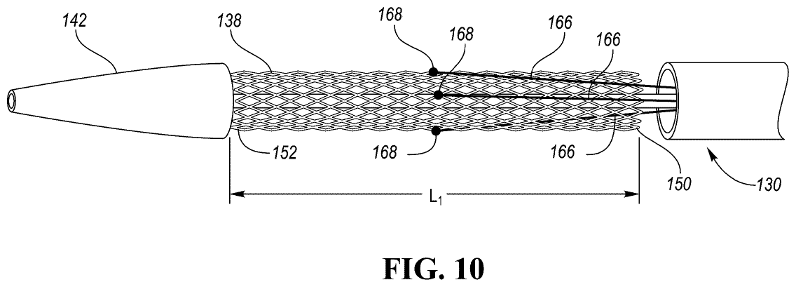

Referring to FIGS. 10-12, the fixation device 138 is generally cylindrical and has a first length L₁ after retraction of the sheath 136. The adjustment members 166 are connected to the attachment points 168 at a point between the proximal and distal ends 150, 152 of the fixation device 138. In operation, actuation, “pulling,” or the like, on the adjustment members 166 towards the catheter portion 130 causes the adjustment member 166 to apply a mechanical force to the fixation device 138 at the attachment points 168. The mechanical force causes the fixation device 138 to translate or shorten (along a longitudinal axis of the fixation device 138) and expand radially outward (i.e., the distal end 152 is translated closer to the proximal end 150 of the fixation device and the diameter of the fixation device 138 increases). For example, as shown in FIGS. 10-12, actuation of the adjustment member 166 towards the catheter portion 130 shortens the fixation device from the first length L₁, to a second length L₂ and/or a third length L₃.

In some embodiments, the adjustment member 166 may be used to elongate the fixation device 138 along its longitudinal axis resulting in the reduction of the diameter of the fixation device 138. For example, actuation or “pushing” of the adjustment members 166 towards the distal tip 142 may increase the length of the fixation device 138 from the third length L₃ to the second length L₂ or the first length L₁, or the like. This allows for the fixation device 138 to be either repositioned, re-adjusted, recaptured by the sheath 136, or the like. In further embodiments, the delivery system 128 includes support members (not shown) for limiting the expansion of the fixation device 138 prior to actuation of the adjustment members 166, limiting the movement of the proximal end 150 of the fixation device during actuation of the adjustment members 166, or the like (e.g., the support members may be connected to the proximal end 150 of the fixation device). Optionally, the adjustment members 166 may be used to support or limit movement or expansion of the fixation device 138 at the proximal end 150, or the like.

For example, in certain embodiments, the plurality of attachment points 168 are disposed at the proximal and distal ends 150, 152 of the fixation device 138. Actuation of the plurality of adjustment members 166 connected the plurality of attachment points 168 disposed at the distal end 152 applies a longitudinal force on the fixation device 138 towards the catheter portion 130 causing the fixation device 138 to translate or shorten and expand radially outward. The plurality of adjustment members 166 connected to the plurality of attachment points 168 disposed at the proximal end 150 can either be used to support and maintain the position of the fixation device 138 or be actuated to apply a longitudinal force on the fixation device 138 towards the distal tip 142. The combination of longitudinal force on the fixation device 138 towards the catheter portion 130 and the distal tip 142 at the distal and proximal ends 152, 150 respectively, shortens fixation device 138 longitudinally and expands the fixation device 138 radially outward. The plurality of adjustment members 166 connected to the plurality of attachment points 168 disposed at the proximal and distal ends 150, 152 of the fixation device 138 may be actuated contemporaneously, separately, individually, or the like, or a combination or sub-combination thereof (e.g., only the plurality of adjustment members 166 connected the plurality of attachment points 168 disposed at the distal end 152 may be actuated, the plurality of adjustment members 166 connected the plurality of attachment points 168 disposed at both the proximal end 150 and distal end 152 may be actuated, or at least one of the plurality of adjustment members 166 may be actuated at one time).

In some embodiments, the adjustment member 166 is connected to an attachment point 168 disposed at the proximal end 150 of the fixation device 138. After the sheath 136 is proximally retracted from the distal end 152 of the fixation device 138, the distal end 152 of the fixation device 138 expands radially outward (by any of the aforementioned means, such as self expansion) to contact the graft 102 and remains in place by radial force, anchors 144, or the like, or a combination or sub-combination thereof. After the sheath 136 is proximally retracted from the proximal end 150 of the fixation device 138 the adjustment member 166 connected to the attachment point 168 disposed at the proximal end 150 is actuated to push the proximal end 150 of the fixation device 138 towards the distal end 152 to shorten the fixation device 138 longitudinally and expand the diameter of the fixation device 138. The expansion of the fixation device 138 radially increases the contact of the fixation device 138 with the graft 102 and the radial force applied by the fixation device 138 to the graft 102 and/or the inner wall of the vessel 118.

In some embodiments, the plurality of attachment points 168 are disposed centrally on the inner surface 156 of the fixation device 138. Actuation of the plurality of adjustment members 166 connected to each of the plurality of attachment points 168 applies an outward radial force to expand the diameter of the fixation device 138. The length of the fixation device 138 decreases as the fixation device 138 expands radially. The fixation device 138 is expanded radially by actuation of the plurality of adjustment members 166 to form a seal between the stent graft 100 and the inner wall of the vessel 118. The fixation device 138 may be self-expanding (as described above) and the radial expansion of the fixation device 138 may be guided, controlled, or assisted by the plurality of adjustment members 166.

In further embodiments, the outer surface 148 of the fixation device 138 may be enclosed by the cover 154, which acts as an external sheath for preventing damage to the vessel 118 during the mechanical adjust of the fixation device 138. The cover 154 may be configured to expand and may mirror the longitudinal and radial adjustments of the fixation device 138. The cover 154 may be proximally retracted from the fixation device 138 at any point, such as prior to the final positioning or full expansion of the fixation device 138, after the fixation device has been fully expanded, or the like. The cover 154 may be proximally retracted into the catheter portion 130. The cover 154 may be composed of any suitable material previously mentioned, or similar to those of the graft 102, the outer shaft 134, or the like. The cover 154 may also include a lubricous layer (such as high density polyethylene HDPE or Polytetrafluoroethylene PTFE) for easier retraction into the catheter portion 130. FIGS. 10-12 show an embodiment with no anchors 144, which may rely on radial force to anchor 144 to the graft 102 and vessel 118 wall. However, as described throughout the disclosure, the fixation device 138 may have anchors 144. The cover 154 and/or catheter portion 130 may prevent the anchors 144 from damaging the vessel 118 wall during tracking of the delivery system 128.

While exemplary embodiments are described above, it is not intended that these embodiments describe all possible forms encompassed by the claims. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, ease of assembly, etc. As such, to the extent any embodiments are described as less desirable than other embodiments or prior art implementations with respect to one or more characteristics, these embodiments are not outside the scope of the disclosure and can be desirable for particular applications.

What is claimed is:

1. A method of deploying a fixation device to anchor a stent graft against an inner surface of a vessel, the method comprising:
positioning a delivery device within the stent graft that has been implanted in the vessel, wherein the fixation device is attached to the delivery device and covered by a sheath;
withdrawing the sheath relative to the fixation device to expose the fixation device; and
expanding the fixation device to apply an outward radial force against an inner surface of the stent graft and an inner surface of the vessel for anchoring and sealing the stent graft against the inner surface of the vessel.

2. The method of claim 1 wherein the expanding step includes self-expansion of the fixation device, wherein the fixation device is composed of a shape memory material that upon withdrawing the sheath returns the fixation device from a radially compressed configuration to an expanded configuration.

3. The method of claim 1 wherein the expanding step includes a balloon connected the delivery device and inflation of the balloon expands the fixation device.

4. The method of claim 1 wherein the expanding step includes mechanically adjusting the fixation device, wherein the delivery device includes a plurality of adjusting members connected to the fixation device and actuation of the plurality of adjusting members expands the fixation device radially by translating the fixation device along a longitudinal axis to shorten the fixation device longitudinally.

5. The method of claim 4 wherein the expanding step includes mechanically adjusting the fixation device by actuation of the plurality of adjusting members shrinks the fixation device radially by translating the fixation device along a longitudinal axis to elongate the fixation device longitudinally for repositioning the fixation device.

6. The method of claim 4 wherein the expanding step includes removing an external sheath surrounding the fixation device before the fixation device applies the outward radial force against the inner surface of the stent graft.

7. The method of claim 4 wherein the fixation device includes a cylindrical metal mesh and the plurality of adjusting members are connected to an inner surface of the fixation device.

8. The method of claim 1 wherein the withdrawing step includes recapturing the fixation device by advancing the sheath relative to the fixation device to cover the fixation device for repositioning the fixation device.

9. The method of claim 1 wherein the fixation device includes an anchor for penetrating the inner wall of the vessel.

10. The method of claim 9 wherein the position step includes inflating a balloon connected to the delivery device for aligning the fixation device within the stent graft such that the anchor is perpendicular to the inner surface of the vessel when the fixation device is expanded.

11. A system for fixating and sealing a prosthesis against an inner surface of a vessel, the system comprising:
a delivery device having a distal end, wherein the delivery device is configured for insertion into a patient's vasculature and advancement of the distal end through the patient's vasculature and into a stent graft that has been deployed in the vessel;
a fixation device attached to the distal end of the delivery device and configured for expanding radially and applying an outward radial force against and the inner surface of the vessel for anchoring and sealing the stent graft against the inner surface of the vessel; and
a plurality of anchors disposed on an outer surface of the fixation device that are configured to penetrate the inner surface of the vessel.

12. The system of claim 11 wherein the fixation device includes a frame having a shape memory configuration with a diameter larger than the diameter of a location within the vessel; and
wherein the fixation device is configured to be compressed and then expanded such that the plurality of anchors are perpendicular to the inner surface of the vessel.

13. The system of claim 12 wherein the frame of the fixation device is composed of nitinol.

14. The system of claim 12 wherein the fixation device includes an inner cover disposed on an inner surface of the fixation device and an outer cover disposed on the outer surface of the fixation device, wherein the inner and outer covers are configured to seal the stent graft against the inner surface of the vessel and guide flow through the stent graft.

15. The system of claim 14 wherein the fixation device includes a proximal end and a distal end distal to the proximal end, and wherein the plurality of hooks are disposed at the proximal end and the distal end of the fixation device.

16. The system of claim 12 wherein the fixation device includes a proximal end and a distal end distal to the proximal end, and wherein the fixation device includes a unitary body having a low profile such that a proximal end and a distal end of the fixation device are in close proximity to each other.

17. The system of claim 11 further comprising:
a balloon configured to inflate and deflate;
wherein the fixation device is disposed on an outer surface of the balloon such that the fixation device expands radially when the balloon is inflated; and
wherein the fixation device is configured to remained expanded after the balloon is deflated.

18. The system of claim 11 wherein the delivery device further includes an adjustment member connected to the fixation device;
wherein the fixation device includes a cylindrical metallic mesh frame; and
wherein actuation of the adjustment members expands the fixation device radially by translating the fixation device along a longitudinal axis to shorten the fixation device longitudinally.

19. The system of claim 18 further comprising an external sheath surrounding the fixation device and configured to be removed from contact with an outer surface of fixation device after expansion of the fixation device, and wherein the external sheath is configured to prevent the plurality of hooks from engaging the inner surface of the vessel.

20. The system of claim 18 wherein the plurality of hooks are disposed between a proximal end and a distal end of the fixation device.
